(19) United States
(12) Patent Application Publication
(10) Pub. No.: US 2023/0404430 A1
(43) Pub. Date: Dec. 21, 2023

(54) AIR BORNE PATHOGENS DETECTION DEVICE

(71) Applicants: Examin Diagnostics Pty Ltd, Birtinya, Queensland (AU); Colin Hickey, Birtinya, Queensland (AU)

(72) Inventor: Colin Hickey, Twin Waters, Queensland (AU)

(21) Appl. No.: 18/031,567

(22) PCT Filed: Aug. 25, 2021
(86) PCT No.: PCT/AU2021/050969

§ 371 Date: Apr. 12, 2023

(30) Foreign Application Priority Data
Oct. 13, 2020 (AU) 2020903711

(51) Int. Cl. A61B 5/08

(52) U.S. Cl. CPC A61B 5/082; A61B 2562/0233; A61B 2560/0431

(57) ABSTRACT

An air-borne pathogen detection device comprises: a testing chamber in which a sensor arrangement is located, a conduit linking an air inlet and the testing chamber so an incoming air is directed toward the sensor arrangement, a return conduit to direct outgoing air toward an air outlet. An optical or electrical quantity across the sensor arrangement varies in dependence of a quantity of airborne pathogens captured in the sensor arrangement. The device comprises or is adapted for connection with a processing means to estimate a quantity of pathogen in the incoming air.

### BACKGROUND OF THE INVENTION

### Technical Field

Embodiments relate to a handheld detection device and, in particular a detection device for detecting the presence of air borne pathogens

### Background

The SARS-CoV-2 virus is creating unprecedented disruption to the world's economy and causing widespread disease. At present there is no known treatment which targets the virus.

Since the SARS-CoV-2 virus primarily infects the lungs, it is readily aerosolised. When an infected patient sneezes/coughs or simply breaths out, moisture droplets containing the virus are launched into the air. Many of these moisture droplets are small enough that they do not settle under the influence of gravity but remain air borne for extended periods (one study suggesting that this virus could be aerosolised for up to sixteen hours).

An essential tool in managing and controlling infections caused by not only this virus, but also other known pathogens, is reliable detection to determine if a person or area is contaminated. There are only three widely-used tests for the SARS-CoV-2 virus currently in use: polymerase chain reaction (PCR) tests, antibody tests and saliva tests

PCR tests are a two-step test. In a first amplification step the viral RNA is replicated to ensure that the presence can be reliably confirmed and once completed (or during the amplification process), the sample is tested to determine if the virus is present. PCR needs time and a carefully controlled environment. Not only does this require a lab, but even in the best-case scenarios, requires a number of hours to produce a result, and most often much longer.

Antibody tests test a patient (typically their blood) to determine if the antibodies produced by the immune system when it encounters the specific virus are present. Since it takes time for the body to produce antibodies, and the antibodies tend to be present even after the infection has cleared, this test is not suitable to determining a risk of infection. Furthermore, this test relates to a person, rather than a place.

Saliva tests are now being approved for emergency use. The COVID-19 saliva test is less invasive and are being developed also for home testing, further these tests for now will require lab work and still require expertise, to ensure the test is accurate. It is a test for individuals and does not detect any airborne pathogens.

### SUMMARY OF THE INVENTION

If a pathogen is detected, the detection device may notify a user.

The notification may comprise an alarm. The alarm may be one or more of visual, auditory and/or an electronic notification such as a text message or an email.

The testing chamber may further comprise one or more settling pads. The settling pad may provide a substrate onto which the pathogen may settle. In an embodiment, the settling pad is comprised of a material which facilitates the detection of the pathogen. The settling pad may be comprised of gold, silver, aluminium or graphene. Other conductive materials may be used.

The settling period may be predetermined by a user or may be pre-set. Where the settling period is pre-set, the settling period may be 5, 10, 15 or 20 minutes. In an embodiment, the detection device will incrementally increase the predetermined settling period at selected intervals until a predetermined maximum is reached.

In one aspect, there is provided an air-borne pathogen detection device, the device comprising a ventilator for creating an airstream, a testing chamber, a conduit linking the ventilator and the testing chamber so that the ventilator can cause air to move into the testing chamber, at least one chamber seal for selectively sealing the testing chamber from effects of the ventilator wherein the testing chamber comprises one or more testing devices for testing the presence of pathogens in the testing chamber.

The one or more testing devices can comprise one or more light sources.

The light sources can be lasers.

The one or more testing devices can comprise one or more detectors.

The detectors can be optical detectors.

The detectors can comprise a filter.

The filter can comprise a long pass filter.

The detectors can comprise antennas.

The antennas can be tuned to a selected wavelength or range or wavelengths.

The selected wavelength or range of wavelengths can be a wavelength or range of wavelengths produced by a selected pathogen when the laser interacts with the selected pathogen.

The antennas can be analogue antennas.

The device can be adapted to detect the presence of a pathogen through Raman spectroscopy.

The device can be adapted to detect the presence of a pathogen through surface-enhanced Raman spectroscopy and/or through tip-enhanced Raman spectroscopy.

The testing chamber can further comprise one or more settling pads.

The settling pad can comprise a substrate.

The substrate can comprise one or more of gold, silver, aluminium or graphene

The detection device can comprise one or more alarms.

The detection device can comprise an inlet connected to the testing chamber so that air can be introduced into the testing chamber via the inlet from a space, wherein the inlet comprises an inlet seal for selectively sealing the testing chamber from the space.

The chamber seal and the inlet seal can be simultaneously operable to substantially seal the testing chamber and prevent movement of air within the testing chamber.

The detection device can comprise a processor connected to machine readable memory, the machine-readable memory storing instructions operate the detection device.

The machine-readable instructions can cause the device to: operate the chamber seal and the inlet seal simultaneously for a predetermined settling period.

The machine-readable instructions can cause the device to: once the predetermined settling period has elapsed, activate the light source and monitor the detector.

In a second aspect, there is provided a method of detecting a pathogen using an air-borne pathogen detection device, the device comprising a ventilator for creating an airstream, a testing chamber, a conduit linking the ventilator and the testing chamber so that the ventilator can cause air to move into the testing chamber, at least one chamber seal for selectively sealing the testing chamber from effects of the ventilator wherein the testing chamber comprises one or more detection devices for testing the presence of pathogens in the testing chamber, the method comprising:
operating the ventilator to cause air to flow into the testing chamber;
operating the chamber seal to seal the chamber;
allowing the air in the chamber to settle for a predetermined settling time;
once the settling time has elapsed, operating the light source and monitoring the detectors to detect an interaction between the light emitted by the light source and the pathogen.

The method can further comprise issuing an alarm if the pathogen is detected.

In a third aspect, disclosed is an air-borne pathogen detection device, the device comprising: a testing chamber in which a sensor arrangement is located, a conduit linking an air inlet and the testing chamber so an incoming air is directed toward the sensor arrangement, and a return conduit to direct outgoing air toward an air outlet. An optical or electrical quantity across the sensor arrangement varies in dependence of a quantity of airborne pathogens captured in the sensor arrangement. The device includes or connects to a processing means to estimate a quantity of pathogen in the incoming air.

The conduit can have a smaller cross-section than the air inlet.

The conduit can be of a smaller cross section than the return conduit.

The sensing arrangement can be located in an air corridor connecting the conduit and an adjacent chamber.

The adjacent chamber, at at least a portion next to the air corridor, can have a larger cross section than the corridor.

The device can comprise an air escape in communication with the adjacent chamber, the escape cavity being adjacent a return wall, wherein air entering into the escape cavity will be guided by the return wall and exit the cavity.

An exit opening for the escape cavity can align with the exit conduit.

The sensor arrangement can be generally positioned above the exit opening.

The device can comprise a filter provided at the exit opening for filtering outgoing airflow into the exit conduit.

The electrical quantity can be an electrical impedance across the sensor arrangement.

The conduit, testing chamber, and return conduit can be located in a removable assembly.

The device can be adapted to provide output from the sensor arrangement to a processing means located away from the removable assembly, wherein the processing means receives electrical signals from a sensing circuit which comprises the sensor arrangement.

Engagement between the removable assembly and the handle assembly can enable an electrical connection between the sensor arrangement and the processing means.

The removable assembly can be a replaceable mouthpiece assembly.

The body assembly can comprise a handle portion and a main body portion.

The handle portion can be angled from the main body portion.

The body assembly can comprise trigger, wherein pressing the trigger will cause a disengagement of the mouthpiece assembly from the body assembly.

In a fourth aspect, there is provided a method of determining a pathogen quantity in an airflow, comprising: receiving an electrical or optical output from a sensing arrangement provided in a device mentioned above, before any airflow is directed toward the sensing arrangement; computing a baseline reading on the basis of the electrical or optical output;
receiving a second electrical or optical output from the sensing arrangement;
computing a test reading on the basis of the second electrical or optical output;
determining the pathogen quantity on the basis of the test reading and the baseline reading.

The method can comprise embedding the data comprising the pathogen quantity into a code and outputting the code.

The code can be a QR code.

Outputting the code can comprise displaying the code.

In a fifth aspect, there is provided computer readable instructions which when executed by a processor, causes the processor to carry out the method mentioned in the aspect or aspects above.

### DESCRIPTION OF THE DRAWINGS

Embodiments are herein described, with reference to the accompanying drawings in which:

FIG. 1 illustrates a cross-section through an air-borne pathogen detection device according to an embodiment;

FIG. 2-1 is a process diagram illustrating a method according to an embodiment;

FIG. 2-2 is a process diagram illustrating a detection phase according to an embodiment;

FIG. 3 illustrates an isometric view of a portable air-borne pathogen detection device according to an embodiment;

FIG. 4 illustrates an exploded view of a disposable mouth piece of an air-borne pathogen detection device according to an embodiment

FIG. 5 illustrates an isometric view of the device shown in FIG. 3, where the body assembly and the mouthpiece assembly are detached;

FIG. 6 illustrates another isometric view of the device shown in FIG. 5, where the body assembly and the mouthpiece assembly are detached;

FIG. 7-1 is an isometric exploded view of a cap located at a front or distal end of the body assembly;

FIG. 7-2 is another isometric exploded view of the distal end cap shown in FIG. 7-1, as viewed from a rear or proximal end;

FIG. 8-1 is an isometric front view of a mouthpiece assembly in accordance with an embodiment;

FIG. 8-2 is an isometric rear view of the mouthpiece assembly shown in FIG. 8-1;

FIG. 9 is an exploded isometric rear view of the mouthpiece assembly shown in FIGS. 8-1 and 8-2;

FIG. 10 is an exploded front isometric view of the mouthpiece assembly shown in FIG. 10-1;

FIG. 11 is an isometric sectional view of the mouthpiece assembly, in accordance with an embodiment;

FIG. 12 is a sectional view of the portable pathogen testing device, in accordance with an embodiment;

FIG. 13-1 is an optical image of a 10×10 array quantum mechanical tunnelling arrangement;

FIG. 13-2 is a close up image of the centre of the sensor shown in FIG. 13-1;

FIG. 13-3 is an electron microscope image of the same arrangement as shown in FIGS. 13-1 and 13-2;

FIG. 13-4 is optical image of an alternative sensor arrangement;

FIG. 14-1 illustrates an isometric rear view of another portable testing device, where the body assembly and the mouthpiece assembly are detached;

FIG. 14-2 illustrates an isometric front view of the device shown in FIG. 14-1, where the body assembly and the mouthpiece assembly are detached;

FIG. 15-1 is an exploded isometric rear view of the mouthpiece assembly shown in FIGS. 14-1 and 14-2;

FIG. 15-2 is an exploded front isometric view of the mouthpiece assembly shown in FIG. 15-2;

FIG. 16 is a sectional view of the portable pathogen testing device shown in FIGS. 14-1 to 15-2;

FIG. 17-1 is a conceptual depiction of a sensing circuit;

FIG. 17-2 is a conceptual depiction of another sensing circuit;

FIG. 17-3 is a conceptual depiction of a further sensing circuit; and

FIG. 18 conceptually depicts a sensing system provided in accordance with an embodiment described by the present disclosure.

### DESCRIPTION OF SPECIFIC EMBODIMENTS

In the following detailed description, reference is made to accompanying drawings which form a part of the detailed description. The illustrative embodiments described in the detailed description, depicted in the drawings and defined in the claims, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

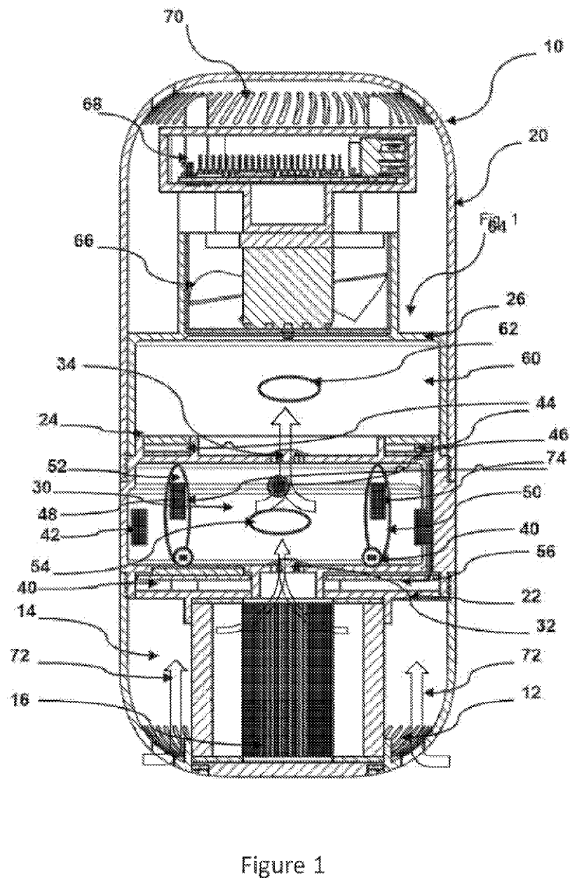

FIG. 1 illustrates an air-borne pathogen detection device 10 according to an embodiment. The device 10 comprises a housing 20 which is divided into a number of chambers by dividers 22, 24 and 26. Divider 22 defines an inlet chamber 14 having a plurality of inlets 12 formed in the bottom and a filter 16. In this embodiment, the filter 16 is a HEPA or Dual N95 filter although other filters could be used in other embodiments.

A testing chamber 30 is defined between divider 22 and 24. The testing chamber 30 includes a port 32 through which air flows from the inlet chamber and a port 34 through which air can flow out of the testing chamber. The ports 32 and 34 further comprises seals (not illustrated) in the form of an umbrella valve which operates as a shutter which can be closed to seal the testing chamber 30 from the inlet chamber 14. In this embodiment, the shutter to seal chamber 34 comprises an inlet seal and the shutter to seal ports 32 comprises an inlet seal.

The testing chamber 30 comprises four substrates 40 which are used as settling pads, as described in further detail below (only two of the substrates 40 are illustrated in FIG. 1) and an array of lasers 44. In this embodiment, the substrates are of the form used in surface-enhanced Raman spectroscopy. For example, the substrates may comprise metal nanoparticles. Other suitable substrates are also known such as Graphene. Such substrates may have the advantage that they do degrade as readily as biological and other reagents which may be required for

In addition, different sensing technologies may be incorporated into the testing chamber 30 and where required, different substrates may have different compositions, as required.

Mounted to the inner surface of the housing 20 in the testing chamber 30 are two detector arrays 42 and 48. In the embodiment illustrated, detector array 42 comprises a long path filter and a radiation detector for detecting radiation emitted by a pathogen when light emitted by the lasers 44 hits the pathogen. Since the wavelength emitted by the pathogen will be characteristic of the pathogen, the detector can be tuned to a particular pathogen by choosing a detector which is tuned to the particular wavelength concerned. In an embodiment, the detector therefore comprises an antenna tuned to the particular wavelength concerned.

In the embodiment illustrated in FIG. 1, the device 10 further comprises an optical sensor 46. As mentioned, the device 10 may include different sensors. Therefore, the optical sensor 46 detects any luminescence emitted when the radiation from the lasers 44 is incident on the substrates 40. The output from the optical sensor 46 is then processed as described in further detail below.

Two TERS (tip-enhanced Raman spectroscopy) concentrators 50 and 52 extend down from the divider 24 towards the substrates 40 and, in this embodiment, assist in the detection of the selected pathogen in a manner known in the art. It is to be realised however that the TERS concentrators 50 and 52 are optional and other embodiments do not include these elements.

A plasma generator 54 is also located on an inner wall of the housing 20 in the testing chamber. When activated, the plasma generator 54 is able to generate a plasma which may be used to sterilise the testing chamber 24. In an alternate embodiment, an ion generator or a source of UV-C light is used instead. It is to be realised that any combination of a plasma generator, an ion generator, a UV-C light, and other known sterilizing may be used.

The substrates 40 include a charging system 56 and current delivery system to produce a static electric charge (not illustrated). This charging system is reversible so that the substrate (or a region close to the substrate) may be positively charged to attract pathogens (which tend to be negatively charged). Then, during a cleaning cycle, the polarity of the static charge is reversed so that the substrate becomes negative, thereby ejecting the pathogens from the substrate which may assist in cleaning and sterilising the substrate.

A sterilizing chamber 60 is formed between dividers 24 and 26. The sterilizing chamber 60 includes a plasma generator 62 attached to the inner wall of the housing 20. The plasma generator 62 operates in the same manner as the plasma generator 54 to sterilize any pathogens included in the sterilizing chamber as well as the walls of the sterilizing chamber. As before, the sterilizing chamber may be provided with an ion generator as well as, or instead of, the plasma generator 62.

An upper chamber 64 is formed between the divider 26 and the upper part of the housing 20. The upper chamber comprises a ventilator in the form of a fan 66 and computing device 68. Outlets 70 are formed in the upper part of the housing 20 and allow air to exit the device 10. The computing device 68 is connected to the fan 66, lasers 44, detectors 42 and 48, ports in the dividers 22, 24 and 26 as well as the charging system 56 of the substrate 40. Therefore, the computing device controls the operation of the functional parts of the device 10 and operation of the device, as described below, is controlled by the computing device 68.

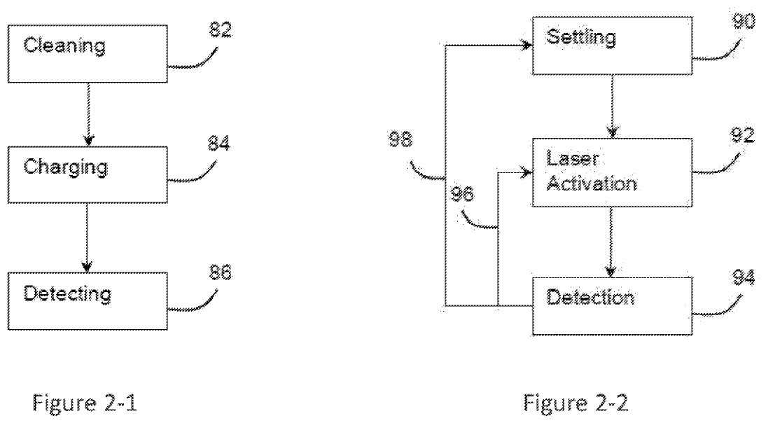

FIG. 2-1 illustrates the basic operation 80 of the device 10. During an initial cleaning phase 82, the polarity of the charging system 56 is reversed to change the polarity of the substrates 40 thereby causing ejection of the pathogens (as described). The plasma generators 54 and 62 are activated to disrupt any pathogens in the testing chamber 30 and the sterilizing chamber 60. The fan 66 is then operated to draw the air containing the now disabled pathogens out of the testing chamber and the sterilizing chamber and expel those disabled pathogens through the outlets 70. Since the pathogens have been disabled they may be safely disposed into the air.

If required, the air can be drawn into the sterilising chamber 60, the ports in the divider 26 closed and the plasma generator 62 operated to sterilize this volume of air. This process may be repeated until the air in device has been sterilized.

At the next step, the charging step 84, the computing device 68 will activate the fan 66 and open the ports in the dividers 22, 24 and 26 to allow air to flow in through the inlets 12 (as depicted by arrows 72 in FIG. 1), through the filter 16, through the open ports in the divider 22 and into the testing chamber 30. The filter 16 will remove dust particles and other aerosols from the air which may interfere with the detection of pathogens. However, the pathogens themselves are small enough to pass through the filter 16 and into the testing chamber 30.

At the same time, the operation of the fan 66 during the charging phase will draw the air that was in the testing chamber into the sterilising chamber 60, as discussed in further detail below.

In the next, phase, the detecting phase 86, any pathogens present in the testing chamber are detected and this phase is described in more detail below with reference to FIG. 2-2.

With reference to FIG. 2-2, the detecting step 86 comprises a number of sub-steps. At step 90, the air in the testing chamber is allowed to settle. This is to ensure that a representative sample of any pathogens for which the test is being run will settle on the substrates 40. Therefore, the predetermined time for the settling period will depend on a number of factors including the precise pathogen being tested for, the volume of the testing chamber, the number and composition of the substrates, the types of detectors and tests used, among others. During this step, the polarity of the charging system 56

Once the pathogens have settled, the tests are run. In the embodiment illustrated, the computing device 68 will operate the lasers 44 at step 92 which are directed towards the substrates 40. Illumination from the lasers 44 will interact with the pathogen (if present) on the substrate and the resulting radiation (optical or otherwise) emitted by the pathogen on interaction with the laser illumination will be detected by the detectors 42, 46 and 48, which are connected to the computing device 68.

At step 94, the computing device 68 will process the output from the detectors 42, 46, 48 and make a determination as to whether or not the pathogen has been detected, according to predetermined criteria. If a determination has been made that the pathogen is present an alarm is activated. The alarm may take one or more of the following: a sound alarm, a visual alarm, communication including email, SMS, notifications etc.

Once the detection step 94 has completed, the process may return to step 92 to reactivate the lasers and detect any radiation emitted by the interaction of the illumination of the lasers with the substrates 40.

Alternatively, the process may proceed from the detection step back to the settling step and allow further settling before activating the lasers in step 92. Once the detection step 84 has completed, the process will return to the cleaning step 86 of FIG. 2-1 as described above.

Although not illustrated in the accompanying Figures, the device 10 includes a battery and is therefore portable.

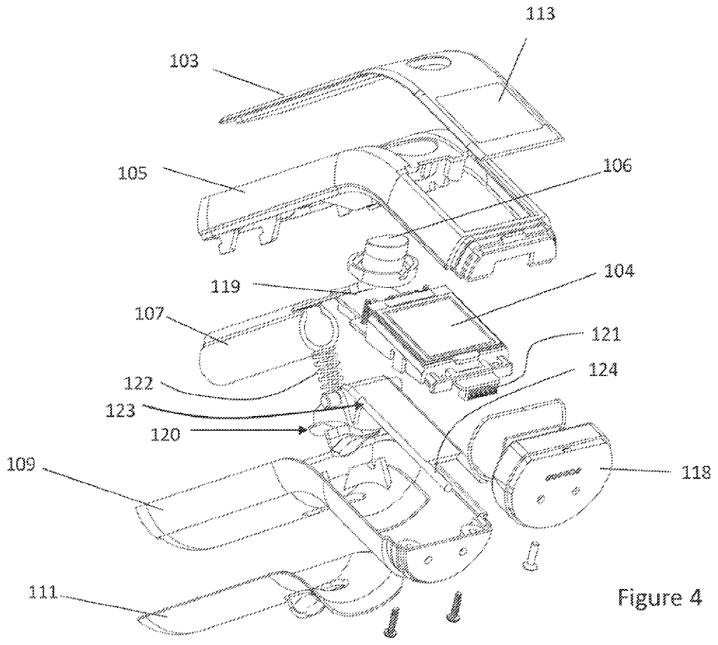

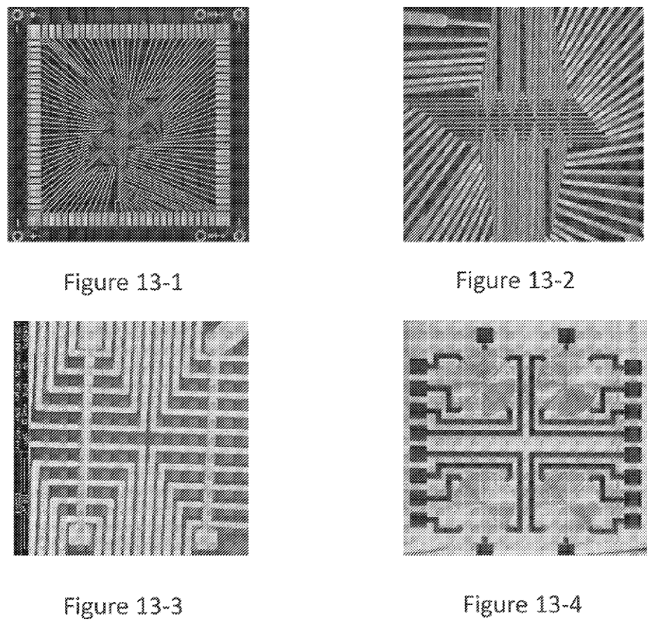

Referring to FIGS. 4 to 12, illustrated is a further embodiment of a portable pathogen detection device 20, configured to implement a method of detecting an amount of air-borne pathogens of a predetermined pathogen type present in a volume of air provided in a breath test. The device 20 may be configured to detect SARS-CoV-2 pathogen, or may be configured to detect another type of pathogen. The volume of air is typically provided by the user or testee blowing into the detection device 100.

The detection device 20 has a removable mouth piece assembly 200 that is sealed from the body assembly 100 of the air-borne pathogen detection device 20. The mouth piece assembly 200 is removable. It may be disposable so that once removed, a new, unused mouth piece assembly 200 is attached pre-assembled onto the body assembly 100 which is reusable. In the illustrated embodiment the air-borne pathogen detection device 20 is in the form of a handheld device, but the technology may be used in other compact or non-compact form factors. Therefore it is preferable to make the mouth piece assembly from relatively inexpensive material.

The body assembly 100 includes a main body portion 101 and a handle portion. The handle portion is angled from the body portion but this is not essential. The main body portion has housed therein a processing module arranged to measure and process the electrical quantity, to calculate the viral load output. The is also housed there in a power source for powering the device. The processing module and the power source are retained in a main body portion 101. The body assembly 100 further comprises a handle 102 adjacent the main body portion 110. A display 104 is provided on the body assembly 100 to show the calculated output. The display 104 may be an LED display. The body assembly 100 further includes a power switch 106 to switch the device on, or on and off. The power switch 106 may be a capacitive switch. In the illustrated embodiment a trigger 108 is provided, where actuation of the trigger will cause the body and mouth piece assemblies 100, 200 to separate. This allows the mouth piece assembly 200 to be removed (e.g., ejected) without requiring to be directly manipulated.

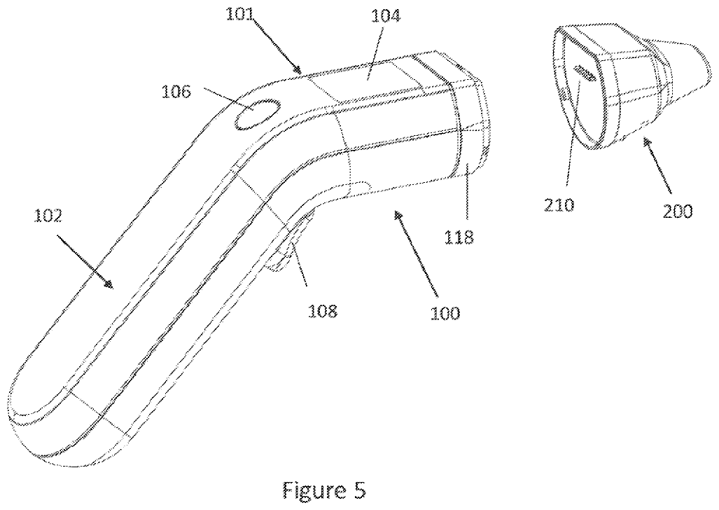

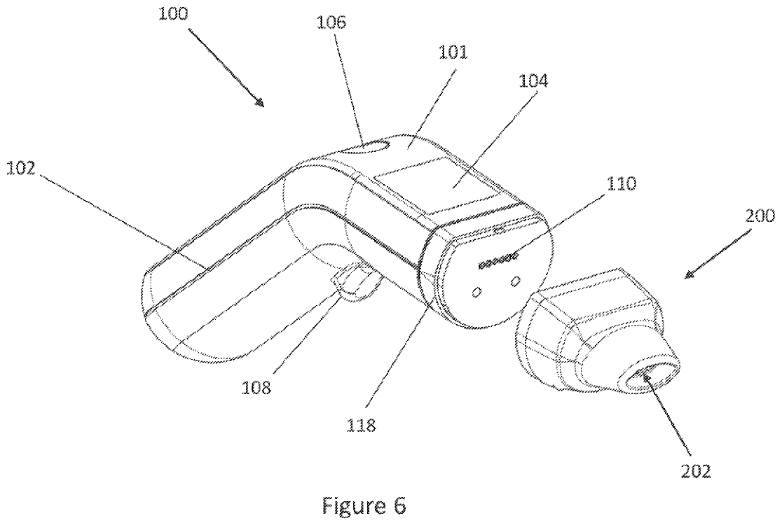

Turning to FIGS. 4-5, the body assembly 100 of the pathogen device 20 is shown according to an embodiment. The body assembly 100 is substantially sealed from the mouth piece assembly 200, with the exception of the connections required between the assemblies 100, 200 to enable electrical connections there between. As can be seen from FIGS. 5 and 6, the connection between the two components is made via contact between sealed electrical pins 210 of the mouth piece assembly 200 and cooperating sealed electrical contacts 110 of the body assembly 100 (see FIG. 6).

The body assembly 100 comprises a handle 102 that allows a user (either the person undertaking the test or a staff facilitating the test) to hold the pathogen device 20 while in use. The handle 102 preferably has an ergonomic design to so that the user can comfortably grasp the handle. The power switch 106 is provided in the form of a button 106 that when depressed will power on the device 10. The button 106 may be capacitive. The device 10 may switch off automatically when it times out from being on and idle for a set period of time, or it can be switched off by depressing the button, or both. The device may further be configured so that the button may be depressed and held in the depressed position to force a reset of the device. A protective shell is provided over the button to seal it from the external environment. In some embodiments, the display 104 is arranged to communicate information to the user when the air-borne pathogen detection device 20 is in operation such as; power on/off status, battery capacity, and an amount of air-borne pathogens of a predetermined pathogen type present. The display 104 is generally disposed on the upper section of the body assembly 100 above the button 106. However, the display 104 may be located elsewhere on the body assembly 100.

Referring to FIG. 6, illustrated is an exploded view of an embodiment of the body assembly 100. The body assembly 100 houses a battery 107 which may be a rechargeable battery, the trigger mechanism 120, the processor 119 which may be a microprocessor, the power button 106 and display 104, which are all disposed inside of the handle 102 and main body portion 101 when assembled. The main body portion 101 and handle 102 are formed by an upper shell 105, together with a lower shell 109. The shells 105, 109 may be moulded from a plastic material. When the upper and lower shells 105, 109 are combined and the aforementioned components (battery, trigger mechanism, button, display and microprocessor) are enclosed, the only exposed component are the electrical connectors 121. The cap 118 is then disposed over these connectors 121 to seal the body assembly from the outside environment but still allows a connection between the processor 119 and the mouthpiece assembly 200. A rubberised seal 111 further encloses the trigger 108 from an underside of the body. A top plastic cover 103 houses a transparent shield 113 that covers the display 104 when the body is assembled.

The trigger 108 of the body assembly 100, when depressed, facilitates the removal of the mouth piece assembly. The trigger 108 is disposed on an underside of the handle 102. The trigger 108 is located adjacent a spring 122. Depressing the trigger 108 biases the spring 122, and removal of the force on the trigger 108 will cause the spring bias to return the trigger 108 to the undepressed position. The spring may be replaced by another resilient deformable. Depression of the trigger 108 actuates the removal mechanism 120. In the depicted case, the trigger 108 is attached to a lever 123. The lever 123 has attached thereto at least one prong 124. The lever 123 and prong(s) 124 are movable by depression of the trigger to cause the removal. The trigger 108 is preferably substantially sealed from the outside environment to avoid entry of pathogens or ingress of other substance such as dirt or moisture into the body assembly 100. To provide the seal, the body assembly 100 has a protective shell 111 is on its underside. The protective shell 111 is flexible at least in the portion covering the trigger 108 so that the trigger 108 can be depressed.

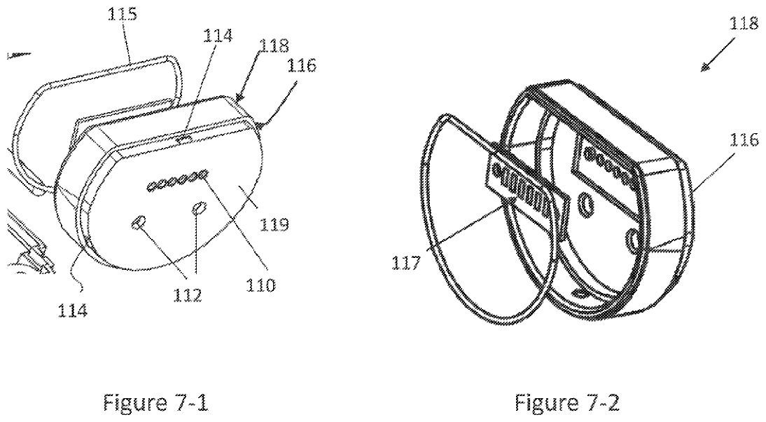

Referring to FIG. 7, a cap 118 is illustrated according to the current embodiment. The cap 118 is disposed at the end of the body assembly 100 which in use will be attached to the mouth piece assembly 200. This end is defined as the distal end of the body assembly 100. A seal 115 may be arranged to seal between the cap 118 and the remainder of the body assembly 100. The cap 118 conforms to the profile of the rest of the body assembly 100 so that it is flush with the rest body assembly 100 when joined thereto. The cap 118 includes means for attaching with the mouth piece assembly 200. It is configured to seal the body assembly 100 to the outside environment whilst still facilitating the connection between the body assembly 100 and mouth piece assembly 200. The cap 118 may be made from copper or a copper alloy containing tin and beryllium. However, the cap 118 may be made of a different material. The different material can be non-metallic, such as plastic. The use of the material helps to destroy or neutralize the pathogen. Other anti-pathogen materials may be used to construct the cap 118. The cap 118 may be entirely constructed from this material. Alternatively, it is only partially constructed of the anti-pathogen material, where the material is used for at least the end face 119 of the cap 118. In further embodiments an anti-pathogen coating to coat at least the end face 119 may be used.

The cap 118 comprises recess or rebate 116 that runs around the perimeter of the end face 119. The rebate 116 is shaped and dimensioned to cooperate with a corresponding skirt of the mouth piece assembly 200, allowing the mouth piece assembly 200 to sit flush against the cap 118 when attached. The attachment is facilitated by cooperating interlocking arrangements provided on the cap 118 and the mouth piece assembly 200, respectively. These may provide a snap-fit or press-fit connection. In the depicted example, a plurality of flexible protrusions 114 (e.g., beads, tabs, hooks, or the like) are disposed around the end face 119. They protrude into the rebate 116. The protrusions 114 are located such that they can be coupled with corresponding notches 214 formed on the mouthpiece assembly 200. The protrusion 114 is configured to engage the notch 214, allowing the two components to interlock, to provide the attachment.

The cap 118 comprises electrical contact ports 110 (see FIG. 7) arranged on the distal face 119 to receive the electrical pins 210 of the mouth piece assembly 200. The ports 110 are electrically connected to the circuitry in the body assembly 100, which may be provided on a printed circuit board (PCB) 117, on which the processor 119 is mounted. The processor 119, which may be a microprocessor, is configured to process signals from the sensing circuit to determine the testing result.

The cap 118 has formed therein at least one hole 112 through the end face 119 of the cap 118. In the depicted embodiment, two holes 112 are provided, but a single hole, or three or more, may be provided instead. The holes 112 are generally disposed below the electrical contact ports 110. The holes 112 are positioned so as to each cooperate with a prong 124 carried by the of the trigger mechanism 120. Upon depressing the trigger 108, the prongs 124 will be moved into position to be received by the holes 112, and will recede or return to the original position when the trigger 108 is released. Pressing the trigger 108 causes the prongs 124 to push against the back of the mouth piece assembly 200 and provide enough force to overcome the ‘snap-engagement’ and disengage the mouth piece assembly 200 from the cap 118.

The mouth piece assembly 200 includes a sensor arrangement for detecting the presence of pathogen in the incoming air. The sensor arrangement is configured to provide a measurable electrical quantity which is changeable in response to the presence of pathogens in the incoming air. The mouth piece assembly 200 is further configured to facilitate the flow of the air, so that the incoming airflow will be guided back outside the assembly 200. Components of the mouth piece 200 assembly will be described below.

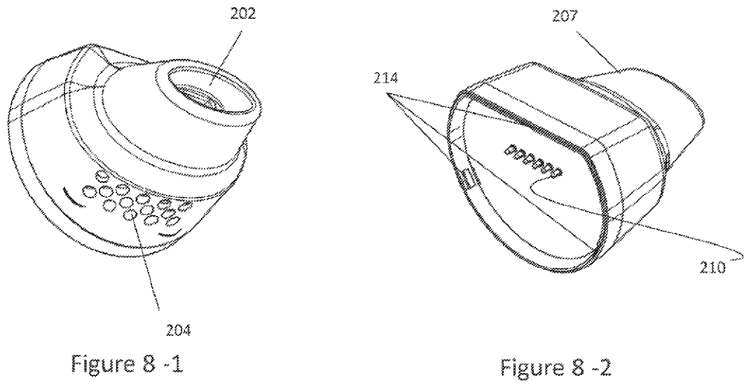

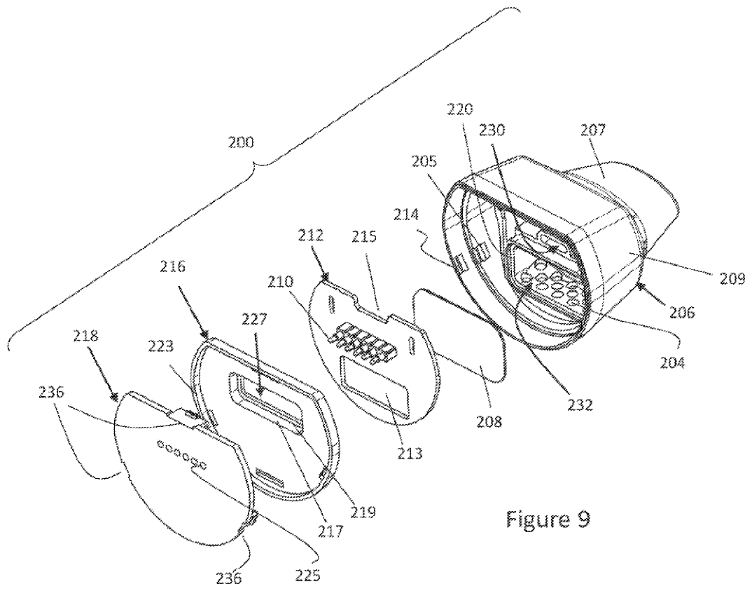

Turning to FIGS. 8-10, the mouth piece assembly 200 of the air-borne pathogen detection device 20 is illustrated according to the current embodiment. The mouth piece assembly 200 comprises a plurality of components as illustrated in FIGS. 9 & 10 that, when combined, form the assembly shown in FIG. 8. When fully assembled as illustrated in FIG. 8, the mouth piece assembly 200 comprises an air inlet 202 and air outlet 204 to facilitate airflow through the mouth piece assembly 200.

Starting from the distal or “front” end, i.e., farthest away from the body assembly 100, the mouth piece assembly comprises: a mouthpiece portion 207, a frame 206 defining air passages, a first partition 212 on which there is located a sensor arrangement 222, a second partition 216 which defines a return surface for the incoming airflow, and a back disk, wall or plate 218 which substantially seals the mouth piece assembly 200 at the proximal or “back” end.

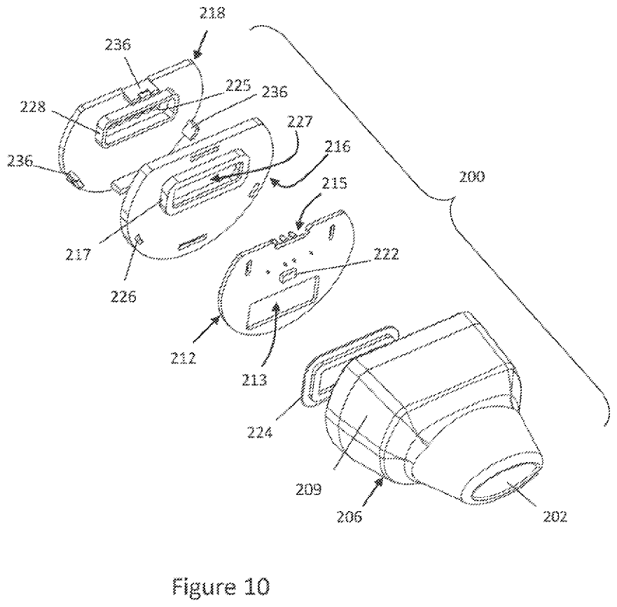

Referring to FIGS. 9-10, the mouth piece assembly 200 comprises a mouth piece portion 207 into which the person being tested will blow. The mouth piece portion 207 is smaller in dimension compared the frame 206. The mouth piece portion 207 defines the air inlet 202 (see FIG. 6) to accept the air blown by the testee. The air inlet 202 is in alignment with a cooperating air passage defined within the frame.

The frame 206 comprises features for cooperating with the body assembly 200, to attach thereto. On an internal surface thereof, the frame 206 comprises a cooperating portion in the interlocking arrangement to create the connection with the cap 118 of the body assembly 100. The cooperating portion is in the form of a plurality of notches 214 which cooperates with the flexible protrusions 114 on the cap 118. The cooperating engagement provides a press fit or snap fit.

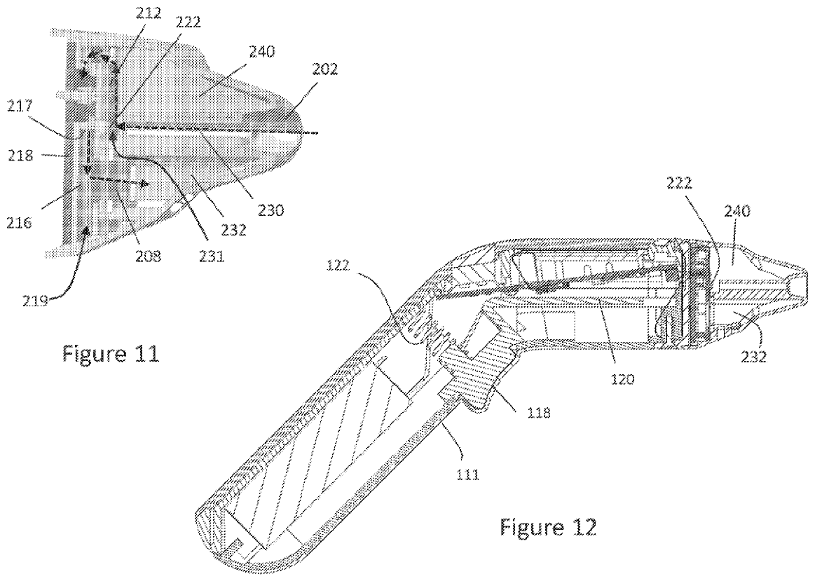

The frame 206 further comprises internal structures for defining airflow passages and for retaining functional components such as filters. As shown, the frame 206 comprises an external wall 209. Supported within the external wall 209 are internal walls which defines a passage 230 for guiding the incoming air toward the sensing circuit. When assembled, the walls surrounding the passage 230 will not reach all the way to the first partition 212 on which the sensor arrangement 222 is located. This ensues the air will be able to exit the passage 230 and escape or scatter into an adjacent chamber 240 (see FIG. 11). Thus, the sensing arrangement 222 is located in a passageway or air corridor 231 formed between the partition 212 and the incoming air passage 230. The corridor 231 is of a small cross section compared with the chamber 240. This also helps to concentrate the airflow in the area around the sensor arrangement.

The frame 206 further comprises internal walls which define a passage 232 for outgoing air. The passages 230, 232 are generally disposed in a vertical relationship to each other. The incoming air passage 230 is located generally above the outgoing air passage 232. The outgoing air passage 232 is in fluid communication with the air outlet 204, which in this case is provided as a plurality of outlet apertures. The outgoing air diffuses out of the apertures.

The incoming air passage 230 is generally smaller in cross section than the outgoing air passage 232. The sizes of the passages can be varied to control the air velocity. The smaller size of the incoming air passage 230 means the relative airspeeds of the air prior to reaching the sensor arrangement (i.e., in the incoming air passage 230) may differ from those of the air after they pass through the sensor arrangement. The larger cross section of the outgoing air passage 232, and the larger cross section of the air outlet 204 (i.e., combined cross sections of the apertures) means the outgoing air will release at a slower speed compared with the air being blown in. The air passages 230, 232 are shown to have generally rectangular shapes. However, other shapes may be used, provided they fit within the physical constraints and provide the required functions of directing the incoming air.

A shield 208 may be arranged to be provided across the outgoing air passage 232, to act as a filter to the volume of outgoing air, before it reaches the outlet 204. The shield 208 comprises or is an air permeable layer which is of material which filters outbound pathogens. The shield 208 may be fitted to the free end of the walls 233 surrounding the outgoing air passage 232. However, it may instead be retained, e.g., on the first partition 212. The layer, in addition or alternative to being made of the aforementioned material, may further be connected to a power arrangement which applies an electrical charge to the film so as to help neutralize or destroy pathogen in the outcoming air.

The first partition 212 is positioned adjacent the shield 208. When assembled, the first partition 212 will be retained within the frame 206. The first partition 212 carries thereon or retains a sensor arrangement 222 (see FIG. 10). The sensor arrangement 222 is located on a distal side of the first partition 212. The sensor arrangement 222 is positioned in the incoming air path so that it faces the incoming air flow being blown into the mouth piece portion 207. Electrical connections to the sensor arrangement are provided through the partition 212, to electrical pins 210 disposed on the proximal side of the partition 212.

The first partition 212 includes or at least partially defines an aperture to allow airflow to exit from the chamber 240 (shown in FIG. 11) and into the space defined between the first and second partitions 212, 216. For instance, the first partition 212, along an upper edge thereof, includes a notch 215. The notch 215 may be a cut out. The notch 215 and the surrounding wall 209 of the frame 206 define an opening, through which air from the chamber 240 can pass, and enter a cavity that is formed between the partitions 212, 216.

The partition 212 further has formed therein an opening 213 located away from the sensor arrangement 222, to facilitate a return path for the air, back from the cavity formed first and second partitions 212, 216. Therefore, the opening 213 is also referred to as a return opening. The return opening 213 is located so that when assembled, it will generally align with the outgoing air passage 232 in the frame 206. In some embodiments, the shield or filter 208 is attached to the partition, to be provided across the opening 215, thus filtering or acting on the air passing through the opening.

The second partition 216 is located adjacent the first partition 212. The second partition 216 is provided with a through aperture 227, for allowing the electrical pins 210 to extend therethrough. The aperture 227 may be generally surrounded by a raised flange or similar structure 217, which protrudes or extends from the distal side of the second partition 216, and when assembled, toward the first partition 212. The skirt or flange 217 may define an opening therein which is slightly smaller than the through aperture 227.

When assembled, the raised flange 217 will generally extend across the separation between the partitions 212, 216, so as to close the gap between the partitions 212, 216. Thus, air coming through the opening or cut out 215 will need to flow around the raised flange 217. In other words, a cavity is defined between the raised flange 217, the surrounding wall 209 of the frame 206, and the two partitions 212, 216. From this cavity, the air can only return back toward the distal end, via the return opening 213 in the first partition 212.

On a proximal side thereof, the second partition 216 further comprises a flange or skirt 223 which is provided substantially around its perimeter. The flange or skirt 223 is configured to be closed around the back plate 218. The back plate 218 and the flange or skirt 223 may have cooperating components for facilitating a snap-fit. When assembled, the back plate 218 will sit flush with the skirt or flange 223. The cooperating components may include slots 226 disposed around the edge of the first partition 212 to retain therein cooperating tabs or latches 236 extending from the back plate 218.

As visible in FIGS. 9 & 10, The back plate 218 has formed therein a plurality of holes 225, each for accepting one of the electrical pins 210. This arrangement helps to seal between the pins. Additional components such as a sleeve or tube structure (not shown) may also be provided to help with electrical insulation between the pins 210.

In some embodiments, the back plate 218 may comprise a raised lip 228 extending on its distal side, around the holes 225. The raised lip 228 may be positioned and dimensioned to fit into the through aperture 227 in the second flange 216. When assembled, the raised lip may be located against the raised flange 217 on the second partition 216, as can be best seen in FIG. 11. This helps to seal against potential air leakage between the second partition 216 and the back plate 218.

The raised edge 228 functions as a sealing lip around the pins 210 to help to protect them against any ingress of dust, moisture, etc.

The airflow through the mouth piece assembly 200 is illustrated in FIG. 11. Air blown into the device 20 by the person being tested will enter the mouth piece assembly via the air inlet 202. From the air inlet 202 the air flows through the incoming air passage 230 and towards the sensor arrangement 222, and then into the air chamber 240 adjacent the passage 230. Continued air supply from the person will displace the air in the chamber 240, which will exit the chamber 240 through the opening or cut out 215 in the first partition 212. The air then enters the cavity 219 between the partitions 212, 216. In the cavity 219, the raised flange 217 of the second partition 216 directs the air to flow around the flange 217 and down towards the lower portion of the cavity 219, preventing any air from passing the second partition 216 towards the body assembly 100. With no other direction to travel the air returns through the air return opening 213 through the first partition 212, and into the outgoing air passage 232. The air then exits from the air outlet(s) 204 from the outgoing air passage 232. Before the air can enter the passage 232 and exit through the outlet 204 it passes through the shield 208, to help to remove the remaining pathogen, if any, in the returning air

The sensing arrangement 222 is arranged to measure the capacitance across the sensor as the inlet air passes over it. The sensor is in the form of a small linear chip that has a plurality of quantum channels arranged to be the same size as the pathogen wished to be tested. It is important to note that different pathogens have different size and electrical quantities, so the sensor has to be arranged for the specific type of pathogen to be tested. The microprocessor 107 determines the quantity of pathogens on the basis of the capacitance that is measured across the sensor when pathogens fall into the quantum channels, attracted by the aptamers, the electrical charge to the sensor, or both.

The processor performs calibration step, to make a ‘baseline’ calculation, when the unit starts up or when the unit is reset. The calculation provides a baseline reading obtained without any air being blown into the device. The baseline reading provides a profile of the background noise or other environmental interference that the sensor detects as baseline readings. During testing operation, the measurements obtained will be calibrated in relation to the baseline. For instance the baseline reading obtained during calibration will be subtracted from the reading obtained during testing.

The sensing arrangement performs optimally in standard conditions i.e. 24° C. but can work in a range of temperatures and has been tested to be operable in 4° C. to 34° C. In preferred embodiments, the unit is configured to generate and display results within a few seconds. In sub-optimal environmental conditions the unit may still be operable, but it may take longer (e.g., up to a minute) for the results to be generated and displayed.

In the hand-held embodiment, there may only be one partition plate instead of two. For instance, the first partition may be modified to include the attachment components for attaching with the back plate. The raised lip or flange extending from the back plate 218 may directly fit over the pins 210 extending from the back of the first partition 212, to seal them from the airflow. The second partition 216 in this case is not required.

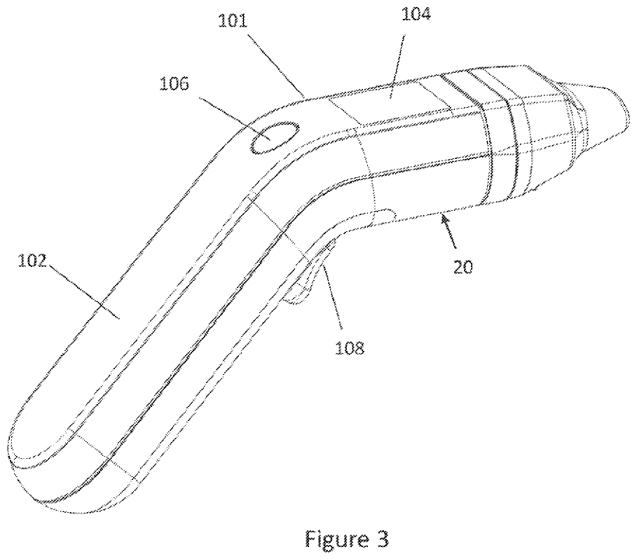

FIGS. 14-1, 14-2, 15-1, 15-2, and 16 depict an example alternative embodiment of the hand-held pathogen testing device. The hand-held pathogen testing device 30 is similar to the device 20 shown in FIGS. 3 to 12, but with a different structure for the mouthpiece assembly and a different airflow. The device 30 utilizes a sensing arrangement and circuitry configured with the same principles as those described in respect of the device 20.

Referring to FIGS. 14-1 and 14-2, the device 30 comprises a body assembly 1400 and a mouthpiece assembly 1450 as in the device 20 of FIGS. 3 to 11. The assemblies 1400, 1450 are adapted to make electrical connection with each other, using respective cooperating electrical contacts.

The body assembly 1400 includes the same components as those shown in respect of the body assembly 100 although it has a slightly varied electrical connection. The body assembly 1400 includes a contact arrangement 1402. The contact arrangement comprises contact pads 1404. The contact pads 1404 are accessible via an opening 1406 formed in the distal (or front) end face 1410 of the end cap 1408. Other components of the body assembly 100 provided in the portable device 20 are also provided in this embodiment. The end cap 1408 provides the same function and has the same general structure as the end cap 118 in the previous embodiment, but with slight variations configured to accommodate the mouthpiece assembly 1450.

FIGS. 15-1 and 15-2 show the mouthpiece assembly 1450 in more detail. Starting from the distal or “front” end, i.e., farthest away from the body assembly 1400, the mouth piece assembly comprises: a mouthpiece portion 1412, a frame 1411 defining air passages and outlets, a partition 1416 on which there is located a sensor arrangement 1418, and a back disk, wall or plate 1420 which substantially seals the mouth piece assembly 1450 at the proximal or “back” end thereof. Here can also be seen that the partition 1416 provides a return surface for the airflow.

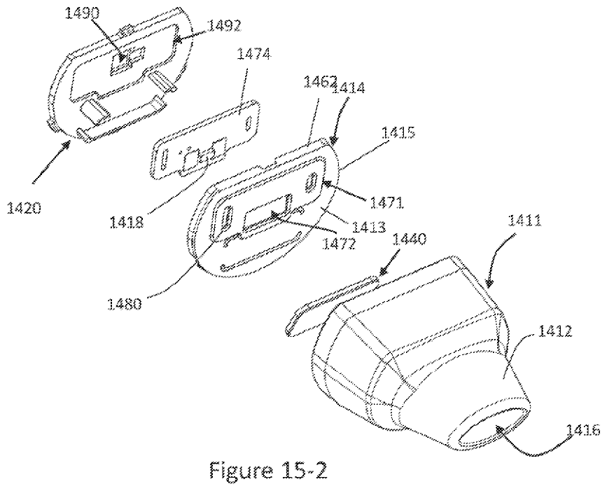

As in the previous embodiment, the mouthpiece portion 1412 is smaller in dimension than the frame 1411. The mouthpiece portion 1412 defines the air inlet 1416 (see FIG. 15-2) to accept incoming air. The air inlet 1416 is in alignment with a cooperating incoming air passage 1432 defined within the frame 1411. As in the previous embodiment, the mouthpiece portion 1412 and the frame 1411 may be provided in one piece, e.g., co-moulded.

The frame 1411 comprises features for cooperating with the body assembly 1400, to attach thereto. On an internal surface thereof, the frame 1411 comprises a cooperating portion in the interlocking arrangement to create the connection with the cap 1408 of the body assembly 1414. The cooperating portion is in the form of a plurality of receiving spaces 1420. The receiving spaces 1420 may be blind or through openings. They may be provided in the form of notches, recesses, grooves, depressions, or the like. The receiving spaces 1420 are located and configured to receive and thus cooperate with the flexible protrusions 1422 on the cap 1408. The protrusions 1422 may be lugs, tabs, bumps, or the like. The cooperating engagement provides a press fit or snap fit. It will be appreciated that receiving spaces 1420 may instead be formed to be accessible from an outer rim of the cap 1408 of the body assembly 1400, with the cooperating protrusions 1422 provided on the internal rim of the frame 1411.

The frame 1411 also comprises features for retaining the aforementioned partition 1414. As shown the features include protrusions such as an arrangement of retaining tabs which are configured to be secured in cooperating notches or receiving openings the partition 1414. The locations of the protrusions 1421 and the cooperating notches or openings 1460 may be reversed.

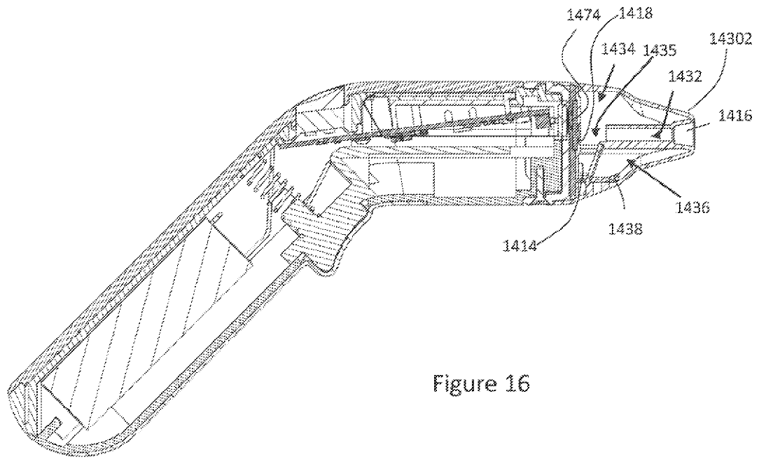

Within the external wall 1430, the frame 1411 comprises internal structures for defining airflow passages and for retaining functional components such as filters. The frame 1411 comprises an external wall 1430. Supported within the external wall 1430 are internal walls which defines the incoming air passage 1432 for guiding the incoming air toward the sensing circuit 1418. When assembled, the walls surrounding the passage 1432 will not reach all the way to the partition 1414. This ensues the air will be able to exit the passage 1432 and escape or scatter into an adjacent chamber 1434 (see FIG. 16).

The frame 206 further comprises internal structures which define an outgoing passage or chamber 1436 for outgoing air. Similar to the embodiment shown in FIGS. 3 to 11, the passages 1432, 1436 are generally disposed parallel to each other, in a vertical orientation to each other. The incoming air passage 230 is located generally above the outgoing air passage 1436. The outgoing air passage 1436 is in fluid communication with the air outlet(s) 1438, provided as a plurality of apertures in communication with the outgoing air passage or chamber 1436.

As in the previous embodiment, the incoming air passage 1432 is generally smaller in cross section than the outgoing air passage 1434. The sizes of the passages can be varied to control the air velocities. Also, the air passages 1432, 1436 may have different cross-sectional shapes than shown, provided the structures fit within the physical constraints and provide the required functions of directing the incoming air. Further, as in the previous embodiment, in this airflow, the cross section of the outgoing air passage 1436, and the combined cross section of the air outlets 1438, are each preferably larger than the cross section of the incoming air passage 1432. Thus, the relative airspeeds of the air prior to reaching the sensor arrangement (i.e., in the incoming air passage 1432) will differ from those outgoing air, which will tend to travel at a slower speed than the air being blown in.

This embodiment also provides a shield 1440, to be arranged to be provided across the outgoing air passage 1432, again, to act as a filter to the volume of outgoing air, before it reaches the outlet 1438. The shield 1440 comprises or is an air permeable layer which is of material which filters outbound pathogens. In this case the layer 1442 is retained within a shield frame or mount 1444. The shield 1440 is fitted to the free end of the walls surrounding the outgoing air passage 1436. As in the previous embodiment, the shield 1440, in addition or alternative to being made of the aforementioned material, may further be connected to a power arrangement which applies an electrical charge to the film so as to help neutralize or destroy pathogen in the outcoming air.

The assembly frame 1432 further comprises, on either sides of the walls of the air passages 1432, 1436, internal walls 1417 intended to cooperate with the partition 1414 to define the air chamber 1434.

The partition 1414 is positioned adjacent the assembly frame 1432. When assembled, the partition 1414 will be retained within the frame 1432, by cooperating locating arrangements. As mentioned this includes notches or depressions 1460 formed in the partition 1414. The notches or depressions 1460 extend internally from a perimeter 1415 of the partition 1414.

On a front or distal face 1413 thereof, the partition 1414 includes a through opening 1472 which extends through the partition 1414. The through opening 1472 may be positioned in a front recessed area 1471, recessed from the front face 1413. The opening 1472 is generally positioned in the partition 1414 such that the opening 1472 will be in general alignment with the incoming air passage 1432. It should be noted that this alignment needs not be perfect or exact, as long as the sensing circuit which will be presented through the opening 1472 will be in position to be in the path of the incoming airflow.

Engagement apertures 1480 may be provided adjacent the through opening 1472, in this cases on either sides thereof. The engagement apertures 1480 are configured to receive therein cooperating formations 1482, such as lugs, tabs, or the like, extending rearwards from the internal walls 1417. These provide a cooperating arrangement to help position the opening 1472 to accept the incoming air flow. The locations of the cooperating engagement apertures 1480 and the formations 1482 may be reversed. Though not shown, a seal may be provided to reduce or prevent air leakage between the internal walls 1417 and the front face 1413 of the partition 1414.

The partition 1414 is configured to retain thereon the sensor arrangement 1418, to be presented through the opening 1472 and toward the incoming air passage 1432. In this embodiment, the partition 1414 comprises a rear recessed area 1470 on its proximal or rear face 1464, so that a mounting plate 1474 on which the sensor arrangement 1418 is mounted may be accommodated therein. The rear recessed area 1470 is aligned with the front recessed area 1471. An engagement arrangement may be provided to secure the mounting plate 1474 in the rear recess 1470, such as a cooperating arrangement of apertures and formations.

The partition 1414 comprises a flange 1462 which extends from the perimeter 1415 of the partition, away from the proximal (or rear) face 1464 of the partition 1414. The flange 1462 is configured to retain the back plate 1420. This is achieved by an arrangement of retaining apertures 1466 on the flange 1462 and cooperating formations such as lugs or tabs 1468 on the back plate 1420, or vice versa.

The back plate 1420 is configured to be received within the flange 1462 of the partition 1414. It will be understood the flange may instead be provided on the back plate 1420, to engage the partition 1414. The backplate 1420 has formed therethrough an access opening 1490 configured for electrical contacts 1419 of the sensor arrangement 1418 to be accessible by the body assembly 1400. The access opening 1490 may be configured to conform to a dimension and shape of at least a part of the sensor arrangement 1418 where output from the sensor arrangement 1418 is to be accessed. The access opening 1490 may be itself provided within a recessed area 1492 (see FIG. 15-2). The recessed area 1492 is preferably generally conforming in shape and dimension to the mounting plate 1474 for the sensor arrangement 1418. When the back plate 1420 is assembled onto the partition 1414, the mounting plate 1474 will be retained within the space defined between the recess 1492 on the back plate 1420 and the rear recess 1470 on the partition 1414.

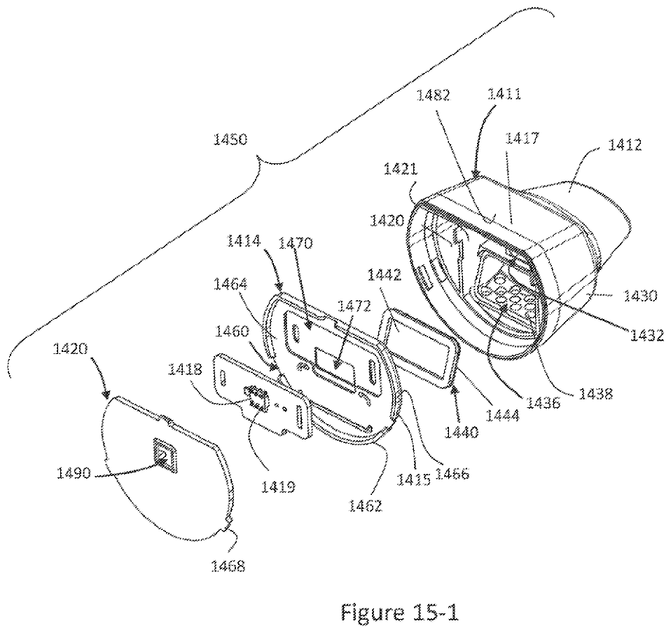

As can be seen in FIG. 14-1, the back plate 1420, on its rear face 1494, carries an attachment flange or skirt 1496 or mounting onto a cooperating rebate 1497 on the end cap 1408 of the body assembly 1400. A cooperating arrangement of engagement features may be provided to facilitate the secure attachment as previously described.

As can be seen in FIGS. 14-1 and FIG. 15-1, on the rear face 1494 of the back plate 1420, around a rim for the access opening 1490 there is provided a protruding lip 1491. The protruding lip 1491 is configured to be received by the opening 1406 through the front face 1410 of the cap 1408. When assembled this will position the exposed contacts 1419 of the sensor arrangement 1418 directly against the cooperating exposed contacts 1404 of the contact arrangement 1402, to form a connection for electrical signals to be conducted therethrough in use.

The embodiment shown in FIGS. 14-1, 14-2, 15-1, 15-2, and 16 utilises electrical connection between contact tabs and pads, whereas the connection shown in FIGS. 3 to 11 is a male (pin)-female (port) connection. However, it will be understood that either connection arrangement, or further variations which provide the same functionality, may be used in the embodiments.

The airflow within the mouthpiece assembly 1450, in operation, will be described with reference to FIG. 16. The incoming airflow will enter the assembly 1450 via the inlet 1406, and flow into the incoming air passage 1432. The incoming air passage 1432 generally directs the air toward the sensor arrangement 1418. The air is then scattered or redirected by one or more of the sensor arrangement 1418, sensor arrangement mounting plate 1474, and the partition 1414. The air may escape or be scattered into the adjacent chamber 1434. The chamber 1434 is generally defined between the partition 1414, and the wall 1430 of the frame 1411. As the outgoing passage 1436 and air outlets 1438 are communication with the chamber 1434, continued incoming air pressure will cause air to exit through the passage 1436 and then outlets 1438.

Here, the sensing arrangement 1418 is located in a passageway or air corridor 1435 formed between the partition 1414 and the incoming air passage 1432. The corridor 1435 is of a smaller cross section compared with the chamber 1434. It may also be of a smaller cross section compared with the exit passage 1436. This arrangement also helps to concentrate the airflow in the area around the sensor arrangement 1418 to help improve the sensitivity of the device 30. In embodiments of the device, various sensor arrangements may be used.

For instance, the optical sensing arrangement discussed in relation to FIGS. 1 and 2 may be utilised in the portable device. For instance, the light source may be disposed on the body assembly for irradiating the sensor arrangement in the mouthpiece assembly. Partitions or walls between the light source and the sensor arrangement will thus need to be made to allow the transmission of the light or wavelength therethrough.

As mentioned above, the portable embodiments have been described to have a sensing arrangement adapted to include a varying capacitance portion, where a capacitance across the portion varies in dependence of the amount of pathogen detected. Sensor arrangements of these type make use of an electrically charged conductive substrate, doped or coated with aptamers specific for binding or attracting the particular type of pathogen whose presence is under test.

To provide sufficient contact surface to detect the pathogen, and for outputting the electrical signal measurable at the processing unit, the sensor arrangement may be implemented by an electrode gap device. The electrical gap is in the form of a quantum mechanical tunnelling gap matched to the size of the pathogen under test, such as the SARS-CoV-2 virus.

In the presence of the virus, the gap is bridged and thus provides an associated change in its capacitance. Such changes are measurable by the electronic measurement circuit and embedded processing unit in the device. The processing is configured to measure the gap device's electrical impedance and compute an amount of the virus detected.

Preferably, an electrical charge is applied across the substrate to assist in the attraction of the pathogen. For example, a positive charge may be applied to attract the SARS-CoV-2 pathogen, as it has a small intrinsic negative charge.

Alternative sensing arrangements providing the above functions may be a graphene substrate, with a series of indentations or wells that same size as the virus, and an aptamer coating and an electrical charge to attract and bind the pathogen.

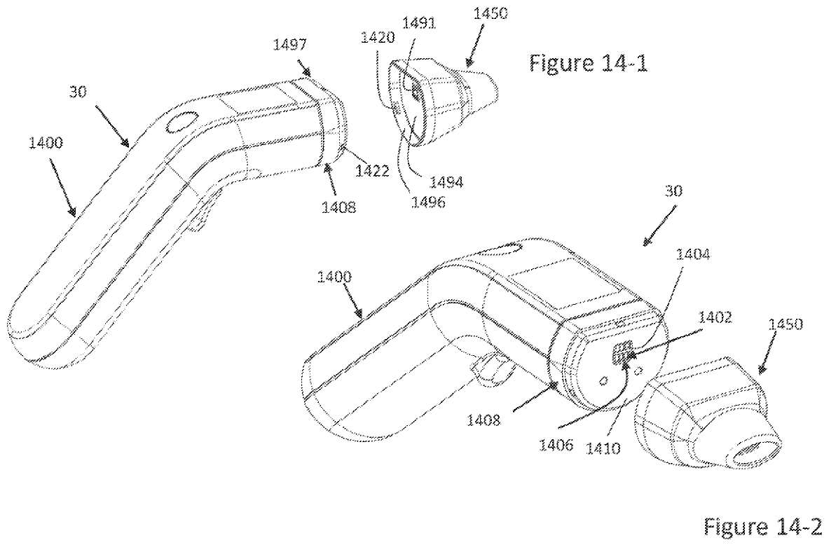

Illustrated in FIGS. 13-1 to 13-3 are an embodiment of the sensor arrangement. FIG. 13-1 is an optical image of a 10×10 array quantum mechanical tunnelling arrangement. FIG. 13-2 showing a close up of the centre of the sensor. FIG. 13-3 is an electron microscope image of the same arrangement as shown in FIGS. 13-1 and 13-2. An optical image of an alternative arrangement is shown in FIG. 13-4. This arrangement comprises the aforementioned tunnels, arranged to form generally concentric rings. This may help to improve the density of the sensing surface, and thus the sensitivity of the device. It will be understood these chips are examples only. Other configurations may be utilised in the sensor arrangement, such as a linear arrangement of reactive portions which are each configured to attract and interact with the trapped pathogens.

The construction of the mouthpiece assembly helps to provide a funnel to concentrate the incoming air and direct it to the sensor arrangement, as the incoming air passage is narrower than the air inlet on to which the person being tested will blow.

Further, in relation to the embodiment shown in FIGS. 14 to 16, the arrangement of the sensor in relation to incoming air passage 1432, the adjacent chamber 1434, and the outgoing passage 1436 further helps to facilitate an airflow, whereby the air will need to pass through the sensing arrangement in order to exit the mouth piece. The portion of the passage where the sensing arrangement 1418 is located is smaller in dimension than the chamber 1434 and also the outgoing passage 1436. This also helps to concentrate the air in the area of the sensor arrangement.

In either portable or non-portable embodiment, applying moisture to the sensing environment, either to where the sensor arrangement is located or to the incoming air, helps to improve the performance of the sensor arrangement as the moisture helps the improve the binding speed for the aptamers to bind to the pathogen. Where provided, air flow generation will apply air pressure toward the sensor arrangement to improve the degree of interaction between potential airborne pathogens and the sensor arrangement.

In embodiments which are breath test devices, separate moisture application is not needed because the incoming breath is already moistened. Also, separate air generation means is not required as the air pressure is supplied by the person blowing into the device.

As mentioned, processing of the signals generated from the sensor arrangement is performed by a processing unit. The sensor arrangement is a part of a sensing circuitry, in conjunction with a power circuit portion to supply the power for the functioning of the sensor arrangement. The processing unit thus receives signals from an overall electrical circuit comprising the sensor arrangement, to process the signals.

For instance, as described above, in some embodiments, the electrical impedance across the sensor arrangement is in dependence of the amount of pathogens which electrically interact with the sensor arrangement. Changes in the impedance can result from changes in capacitance. The capacitance may be measured by determining the capacitance, or more generally, the capacitance, of the sensing arrangement in the sensing circuit.

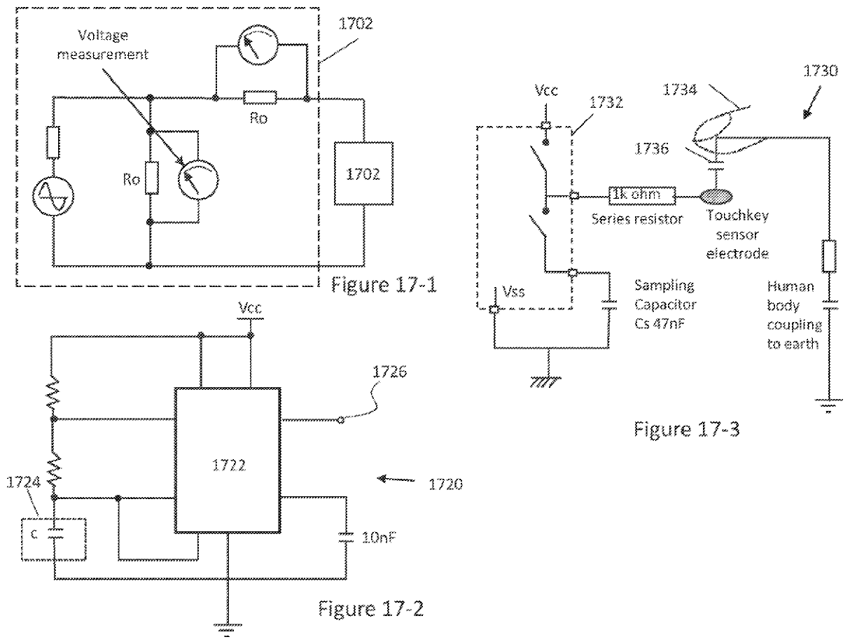

FIG. 17-1 conceptually depicts such a sensing circuit. The sensor arrangement 1702 is the sensing circuit portion where the electrical impedance is under test. The circuitry component 1704 is a remainder of the sensing circuit which may be provided away from the sensor arrangement 1702. For instance, in the portable embodiments, it may be provided in the body assembly, where connection to the sensor arrangement 1702 is facilitated via the connection between the body and the mouthpiece assemblies. In this example configuration the impedance Z at sensors 1702 can be computed using current-voltage measurements using the equation Z=V/I, and the capacitance may be measured using the equation C=1/(jwZ), where w is the frequency applied to the sensing circuit.

FIG. 17-2 conceptually depicts another sensing circuit 1720 for sensing the capacitance across the sensor arrangement. In this example, a timer circuit 1722 is used to drive the power through the sensing circuitry 1720. By measuring the output frequency at the output 1726, the capacitance at the sensing arrangement 1724 can be computed as C=1/(2*pi*P′R), where f is the output frequency, and R is the resistance value of each resistor.

FIG. 17-3 conceptually depicts another sensing circuit 1730 which uses a capacitive touch capable controller 1732, for measuring the signal from the sensor circuit 1736 when capacitive touch, e.g., from a user's finger 1734, is made. This embodiment has a lower cost but likely lower accuracy.

In an example implementation, the sensor arrangement is driven by a driving circuit which provides an electrical signal of a particular frequency. For instance, to generate the sensor output, the driving circuit may be configured to provide a square pulse (0-3.3 V with 50% duty cycle and 500 kHz frequency) is applied, generated by the microprocessor 119. In the presence of the virus the capacitance of the electrode gap changes. The device will preferably have a sensitivity to measure the capacitance down to 0.0015 pico-Farads.

As the sensor detects the whole virus, the output is determined by; the virus size (˜125 nm for SARS-CoV-2), binding affinity, and the electrical properties of the pathogen in the sensor's tunnelling gap. Dust particles and other nanoparticles such as exosomes that exist in any saliva droplets that may be directed toward the sensor arrangement have different diameters, and will not bind with the sensor aptamers.

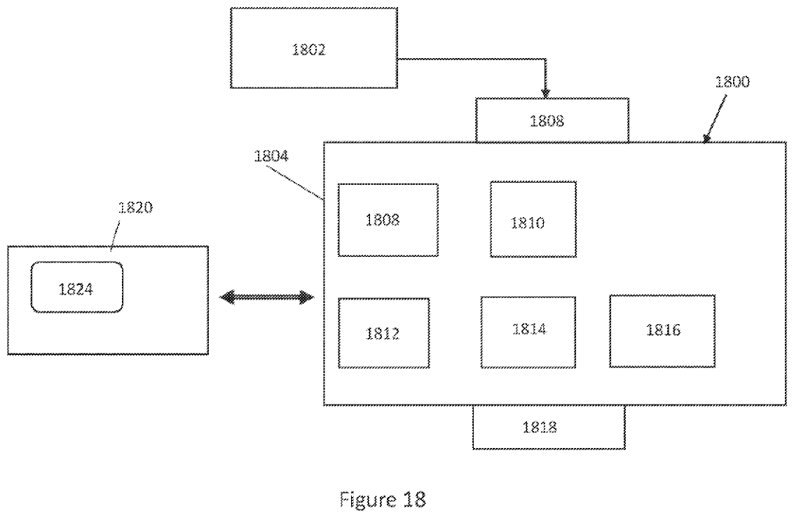

FIG. 18 conceptually depicts a sensing system 1800 comprising a sensor arrangement 1802 and a controller 1804, for detecting a quantity of airborne viruses. The controller 1804 may be provided in the same housing as the sensor arrangement. The controller 1804 may alternatively be provided in a separate housing assembly, from which a housing or assembling containing the sensor arrangement is detachable. The sensor arrangement 1802 is in connection with a signal receiving portion 1806 for providing input to the controller 1804. In electrical sensing embodiments the portion 1806 are electrical connection arrangements such as connection pins, ports or contacts. In optical embodiments where the sensing arrangement functions by outputting a wavelength, the portion 1806 may comprise an optical sensing arrangement.

The controller 1804 comprises a processing module 1808 configured to perform the computations for analysing the sensor arrangement output and to determine the viral quantity detected. The controller 1804 further comprises a power module 1810 for powering the sensing system 1800. A display module 1812 is provided to provide display of data, such as test date, test location, test output, etc. There may be an input module 1814 for the user to provide control or data input. The input and display modules may be a combined interface module. A local memory 1816 is provided to store data such as test information and test results. The controller 1804 may be adapted to provide data output, e.g., via an externally accessible port 1818, such as a USB (universal serial bus) port. This provides a wired transfer of data.

Embodiments of the system may further include a communication module for wirelessly outputting the test data to a remote location, e.g., via a long range communication network or a short range communication network, or both. However, some embodiments do not have wireless connectivity, to prevent hacking attempts and data privacy issues.

The processing unit may be further configured to configured to generate a code that has the test result embedded therein. The embedded data may also include information regarding the test such as test date, test location, test device serial number, etc. The code may be displayed via the display module 1812 to, e.g., an LCD or OLED screen. The code may be read or imaged from a separate device 1820 running a native application or a web-based application 1824, for decoding by a computing or processing unit of the separate device, so as to obtain the embedded information using the separate device 1820. The separate device may be a mobile device such as a smart phone, tablet, portable computer, or another device with the required scanning or imaging, and decoding, capabilities.

Variations and modifications may be made to the parts previously described without departing from the spirit or ambit of the disclosure.

For example, in relation to the portable hand-held embodiments, the internal components may be located within the body assembly in a different manner than that described. For instance, the power module may be provided in the handle.

Also, the body assembly of the portable embodiments does not need to have a handle and a main body which are visible discernible from each other (e.g., as shown in FIGS. 6, 14-1, and 14-2). For instance, the body assembly may provide a combined main body and handle.

Although the hand-held embodiments shown include a trigger whereby pressing the trigger will eject the mouth piece, another removal mechanism may be provided. For instance the mouth piece may simply be snapped or screwed onto the body assembly.

Processing of the measurement results may be done remotely rather than by a processing chip or module inside the device. The measurements may be directly transmitted to the remote processing means (e.g., a server, or a processor in a connected or paired computing device). Even where the processing is done on the device, the detection device need not necessarily have built in displays. Rather the results or both, may be provided to a remote computing device. These variants require connectivity can be via a wired connection or a wireless connection. The wireless embodiments require the device to have built in long range (e.g., mobile data or WiFi) or near range (e.g., Bluetooth®) capability to do so.

Also, in embodiments using electrical pin-port connections, the electrical pins may be provided by the mouth piece assembly for connection into cooperating ports on the body assembly, or the locations of the pins and ports can be reversed. Further alternatively, some of the pins may be provided on the body assembly with cooperating ports on the mouth piece assembly, and some of the pins may be provided on the mouthpiece assembly with cooperating ports on the body assembly.

It is to be understood that, if any prior art publication is referred to herein, such reference does not constitute an admission that the publication forms a part of the common general knowledge in the art, in Australia or any other country.

In the claims which follow and in the preceding description, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments. Similarly, the word “device” is used in a broad sense and is intended to cover the constituent parts provided as an integral whole as well as an instantiation where one or more of the constituent parts are provided separate to one another.

1. An air-borne pathogen detection device, the device comprising:
a testing chamber in which a sensor arrangement is located,
a conduit linking an air inlet and the testing chamber so an incoming air is directed toward the sensor arrangement,
a return conduit to direct outgoing air toward an air outlet, the return conduit being separate from the conduit for the incoming air;
wherein an electrical quantity across the sensor arrangement varies in dependence of a quantity of airborne pathogens captured in the sensor arrangement; and
a processing means to estimate a quantity of pathogen in the incoming air.

2. The device of claim 1, wherein the conduit has a smaller cross-section than the air inlet.

3. The device of claim 1, wherein the conduit is of a smaller cross section than the return conduit.

4. The device of claim 1, wherein the sensing arrangement is located in an air corridor connecting the conduit and an adjacent chamber.

5. (canceled)

6. The device of claim 4, comprising an air escape cavity in communication with the adjacent chamber, the escape cavity being adjacent a return wall, wherein air entering into the escape cavity will be guided by the return wall and exit the cavity.

7. The device of claim 6, wherein an exit opening for the escape cavity aligns with the return conduit.

8. The device of claim 7, wherein in use, the sensor arrangement is generally positioned above the exit opening.

9. The device of claim 7, further comprising a filter provided at the exit opening for filtering outgoing airflow into the return conduit.

10. The device of claim 1, wherein the electrical quantity is an electrical impedance across the sensor arrangement.

11. The device of claim 1, wherein the conduit, testing chamber, and return conduit are located in a removable assembly.

12. The device of claim 11, adapted to provide output from the sensor arrangement to a processing means located away from the removable assembly, wherein the processing means receives electrical signals from a sensing circuit which comprises the sensor arrangement.

13. The device of claim 12, wherein engagement between the removable assembly and a body assembly enables an electrical connection between the sensor arrangement and the processing means.

14. The device of claim 11, wherein the removable assembly is a replaceable mouthpiece assembly.

15. The device of claim 13, wherein the body assembly comprise a handle portion and a main body portion.

16. The device of claim 15, wherein the handle portion is angled from the main body portion.

17. The device of claim 15, wherein the body assembly comprises trigger, wherein pressing the trigger will cause a disengagement of the mouthpiece assembly from the body assembly.

18. A method of determining a pathogen quantity in an airflow, comprising
receiving an electrical or optical output from a sensing arrangement provided in a device in accordance with claim 1, before any airflow is directed toward the sensing arrangement;
computing a baseline reading on the basis of the electrical or optical output;
receiving a second electrical or optical output from the sensing arrangement;
computing a test reading on the basis of the second electrical or optical output;
determining the pathogen quantity on the basis of the test reading and the baseline reading.

19. The method of claim 18, further comprising embedding the data comprising the pathogen quantity into a code and outputting the code.

20. The method of claim 19, wherein the code is a QR code.

21. (canceled)

22. A non-transitory computer readable medium comprising, computer readable instructions which when executed by a processor, cause the processor to carry out the method of claim 18.
